(19) United States
(12) Patent Application Publication
(10) Pub. No.: US 2026/0224841 A1
(43) Pub. Date: Aug. 6, 2026

(54) SYSTEM FOR ASSEMBLING FLEXIBLE ELONGATE INSTRUMENTS

(71) Applicant: Auris Health, Inc., Santa Clara, CA (US)

(72) Inventors: John Bernard Roumbanis, Fremont, CA (US); Krishanu Tapan Das, San Jose, CA (US); Sabrina Lynn Fraser, Sunnyvale, CA (US)

(73) Assignee: Auris Health, Inc., Santa Clara, CA (US)

(21) Appl. No.: 19/044,009

(22) Filed: Feb. 3, 2025

(51) Int. Cl. A61M 25/00

(52) U.S. Cl. CPC A61M 25/0009

(57) ABSTRACT

This disclosure provides a system and apparatus for assembling medical instruments. The present implementations more specifically relate to laser welding an elongate shaft that is longer than the enclosure of the laser welding system. In some aspects, a laser welding enclosure may be coupled to (or include) a light occluding channel that provides a tortuous path through which a flexure can enter or otherwise access the enclosure without allowing any light to escape from inside the enclosure. The light occluding channel may be formed in an opaque or diffuse fixture coupled to or disposed within an opening of the enclosure. The fixture includes a first opening facing outside the enclosure and a second opening facing inside the enclosure, where the openings are connected via the light occluding channel. The shape of the channel is configured to prevent any light incident upon the second opening from exiting through the first opening.

### TECHNICAL FIELD

This disclosure relates generally to medical systems, and specifically to systems for laser welding flexible elongate instruments.

### DESCRIPTION OF RELATED ART

Many medical procedures, such as laparoscopy, ureteroscopy, or percutaneous nephrolithotomy (PCNL), involve a series of complex steps that require careful movement and positioning of medical tools or instruments inside a patient's body. For example, to remove urinary stones from the kidney and ureter, a physician can insert a ureteroscope into the urinary tract through the urethra. A ureteroscope includes an endoscope at its distal end configured to enable visualization of the urinary tract. Generally, during a percutaneous access procedure (such as PCNL), the physician (or a technician) drives a needle into the patient, through a target location on the kidney, and uses another medical instrument (which may be in conjunction with the needle) to extract the stone from the kidney via the percutaneous access point.

Many of the instruments or tools used in medical procedures are assembled from multiple component parts. Adhesives are often used in existing assembly processes to attach the component parts to one another. However, the surfaces of the component parts in contact with one another are often very small. As such, the application of adhesives to such small parts can be time consuming and difficult to control. Thus, there is a need to improve the efficiency and robustness of the instrument assembly process.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a laser welding system. The laser welding system includes an enclosure having an opening to receive an elongate shaft, a light occluding apparatus coupled to the opening of the enclosure, and a laser configured to emit a concentrated beam of light inside the enclosure. The light occluding apparatus is configured to provide access to the enclosure for the elongate shaft and prevent the light from the laser from escaping through the opening while a distal portion of the elongate shaft is disposed within the enclosure and a proximal portion of the elongate shaft is outside the enclosure.

Another innovative aspect of the subject matter of this disclosure can be implemented in a light occluding apparatus for a laser welding system. The light occluding apparatus includes a first opening configured to face outside an enclosure of the laser welding system, a second opening configured to face inside the enclosure of the laser welding system, and a channel connecting the first opening to the second opening. The channel of the light occluding apparatus has a shape that prevents any light incident upon the second opening from exiting through the first opening.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows a schematic view of an example elongate instrument, according to some implementations.

FIG. 3A shows a perspective view of an example elongate instrument, according to some implementations.

FIG. 3B shows a cross-sectional side view of the elongate instrument of FIG. 3A.

FIG. 4 shows an example laser welding system, according to some implementations.

FIGS. 5A and 5B show cross-sectional views of an example light occluding apparatus, according to some implementations.

FIG. 6 shows another cross-sectional view of an example light occluding apparatus, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, many of the instruments or tools used in medical procedures are assembled from multiple component parts. Adhesives are often used in existing assembly processes to attach the component parts to one another. However, the surfaces of the component parts in contact with one another are often very small. Thus, the application of adhesives to such small parts can be time consuming and difficult to control. By contrast, lasers can be used to spot-weld small, localized regions in a minimal amount of time.

A laser welder uses a highly concentrated beam of light to fuse two (or more) surfaces together. The laser beam must have a very high intensity to generate enough heat for fusing objects together. Even incidental exposure to such high intensity light (such as due to specular or diffuse reflections) can cause damage to the skin and/or eyes. As such, laser welding is often performed inside an enclosure that seals or traps the laser light therein and/or attenuates the intensity of any light transmitted through the enclosure to relatively safe levels.

Some medical instruments, such as steerable catheters, include flexible elongate shafts (also referred to as “flexures”) that are much longer than the enclosures of many laser welding systems. As a result, a laser welding operation can be performed on a portion of the flexure disposed inside the enclosure while at least some of the flexure lies outside the enclosure. Aspects of the present disclosure recognize that a flexure can bend and deform around curves and/or corners that would otherwise occlude or obstruct any incident rays of light.

In some aspects, a laser welding enclosure may be coupled to (or include) a light occluding channel that provides a tortuous path through which a flexure can enter or otherwise access the enclosure without allowing any light to escape from inside the enclosure. The light occluding channel may be formed in an opaque or diffuse fixture that is configured to be coupled to or disposed within an opening of the enclosure. The fixture includes a first opening facing outside the enclosure and a second opening facing inside the enclosure, where the first and second openings are connected via the light occluding channel. The shape of the light occluding channel (or tortuous path) is configured to prevent any light incident upon the second opening from exiting through the first opening.

Particular implementations of the subject matter described in this disclosure can be implemented to realize one or more of the following potential advantages. The light occluding channel of the present disclosure provides a relatively low-cost solution for laser welding elongate instruments that are longer than existing laser welding enclosures without significantly expanding the footprints of such enclosures. By providing a tortuous path through an opaque or diffuse fixture, the light occluding channel allows a flexure to be partially inserted into the enclosure (such that at least part of the flexure remains outside the enclosure) while concurrently preventing any harmful rays of light from escaping through the channel. As such, aspects of the present disclosure can improve the safety, efficiency, and robustness of medical instrument assembly processes.

Although certain aspects of the present disclosure are described in detail herein in the context of renal, urological, or nephrological procedures, such as kidney stone removal and treatment procedures, it should be understood that such context is provided for convenience and clarity, and the concepts disclosed herein are applicable to any suitable medical procedure. However, as mentioned, description of the renal or urinary anatomy and associated medical issues and procedures is presented herein to aid in the description of the concepts disclosed herein. In some implementations, the techniques and systems described herein are discussed in the context of a percutaneous procedure, which can include any procedure where access is gained to a target location by making a puncture or incision in the skin, mucous membrane, or other body layer. However, it should be understood that these techniques and systems can be implemented in the context of any medical procedure involving irrigation and/or aspiration of any type of fluid (such as saline solutions and/or blood) via a percutaneous access instrument.

The present disclosure provide systems, devices, and methods for assembling articulable medical instruments. Articulation of instruments in accordance with the present disclosure can be implemented by tensioning one or more tendons, referred to herein as “pull wires,” that traverse a shaft of the instrument. The term “pull wire,” as used herein, can refer to any type of cable, cord, strand, tendon, filament, rod, band, tether, wire, string, fiber, chain, line, strap, tape, tube, lead, ribbon, or the like, configured to transmit force from an articulation control driver/actuator to cause articulation of a shaft. With respect to medical instruments described in the present disclosure, the term “instrument” is used according to its broad and ordinary meaning and may refer to any type of tool, device, assembly, system, subsystem, apparatus, component, or the like. In some contexts herein, the term “device” may be used substantially interchangeably with the term “instrument.” Furthermore, the term “shaft” is used herein according to its broad and ordinary meaning and may refer to any type of elongate cylinder, tube, scope (e.g., endoscope), prism (e.g., rectangular, oval, elliptical, or oblong prism), wire, or similar, regardless of cross-sectional shape. It should be understood that any reference herein to a “shaft” or “instrument shaft” can be understood to possibly refer to an endoscope.

During certain procedures, medical instrument(s), such as robotically controlled medical instrument(s) (e.g., endoscopes, access sheaths, working instruments), is/are inserted into a subject (e.g., a patient's body) and articulated or otherwise controlled. Within the subject, the instrument(s) may be positioned within a luminal network or other anatomy of the patient. As used herein, the term “luminal network” refers to any cavity structure within the body, whether comprising lumens or branches (e.g., a plurality of branched lumens, as in the lungs or blood vessels) or a single lumen or branch (e.g., within the urinary or gastrointestinal tracts). Instruments associated with aspects of the present disclosure can include, for example, any type of endoscope (i.e., “scope”), such as a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), or borescope, among other examples.

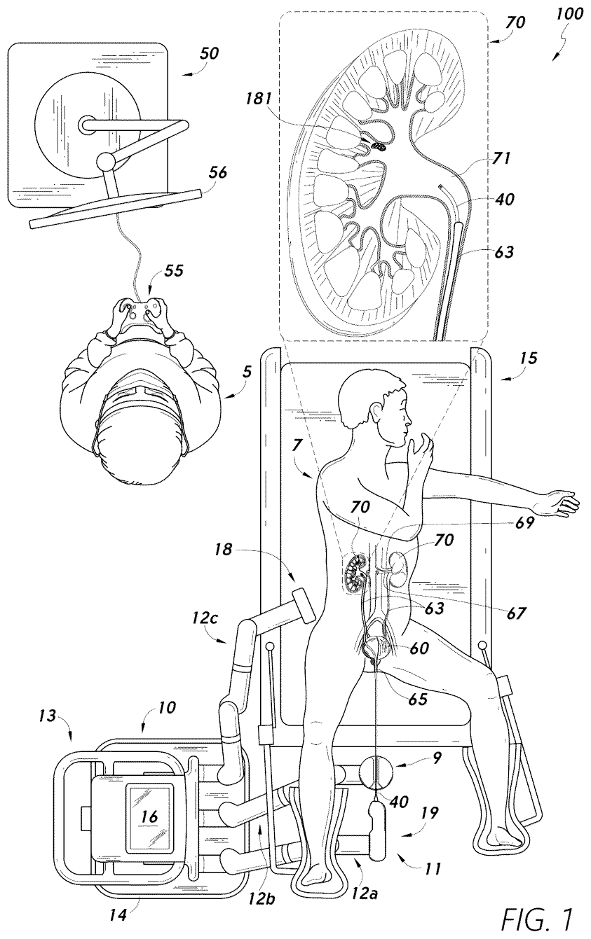

FIG. 1 shows an example medical system 100, according to some implementations. Generally, robotic solutions can provide relatively higher precision, superior control, and/or superior hand-eye coordination with respect to control of certain instruments compared to strictly-manual solutions.

The medical system 100 includes a robotic system 10 configured to engage with and/or control an instrument 19 (e.g., endoscope/ureteroscope) including a proximal base 11 (e.g., handle) and a shaft 40 coupled to the base 11 at a proximal portion thereof. The robotic system 10 can be configured to facilitate execution of a medical procedure and can be arranged in a variety of positions and configurations, depending on the procedure. The medical system 100 can include a table 15 configured to hold the subject patient 7.

The robotic system 10 can include one or more robotic arms 12 configured to engage with and/or control the instrument 19 to perform one or more aspects of a procedure. As shown, each robotic arm 12 can include multiple arm segments coupled to joints, which can provide multiple degrees of movement/freedom. The robotic system 10 can have an instrument feeder instrument 9 coupled to an arm 12b thereof to facilitate robotic advancement of the instrument 19. Another arm 12a may have the instrument base 11 coupled thereto.

The robotic system 10 can be electrically and/or communicatively coupled to any component of the medical system 100, such as to a control system 50, the table 15, an electromagnetic (EM) field generator 18, and/or the instrument 19. For example, the robotic system 10 may be configured to receive control signals from the control system 50 to perform certain operations, such as to position one or more of the robotic arms 12, manipulate (e.g., advance, articulate) the instrument 19. In response, the robotic system 10 can control various components of the robotic system 10 to perform the operations. In some embodiments, the robotic system 10 and/or control system 50 is/are configured to receive images and/or image data from the instrument 19 representing internal anatomy of the patient 7.

The robotic system 10 can include a support structure 14 (also referred to as a “column”) and a console 13 at the top of the column 14. The column 14 may include one or more arm supports (also referred to as a “carriage”) for supporting the deployment of the one or more robotic arms 12 (three shown in FIG. 1). The console 13 can provide both a user interface for receiving user input and a display 16 (e.g., screen or a dual-purpose device such as, for example, a touchscreen) to provide the physician/user 5 with both pre-operative and intra-operative data. Example pre-operative data may include pre-operative plans, navigation and mapping data derived from pre-operative computerized tomography (CT) scans, and/or notes from pre-operative patient interviews. Example intra-operative data may include optical information provided from the tool, sensor and coordinate information from sensors, as well as vital patient statistics, such as respiration, heart rate, and/or pulse.

Articulation of the shaft 40 of the instrument 19 may be controlled robotically, such as through operation of a robotic manipulator of the robotic system 10. The term “end effector” is used herein according to its broad and ordinary meaning and may refer to any type of robotic manipulator device, component, and/or assembly. In implementations in which an adapter, such as a sterile adapter, is coupled to a robotic end effector or other robotic manipulator, the term “end effector” may refer to the adapter (e.g., sterile adapter), or any other robotic manipulator device, component, or assembly associated with and/or coupled to the end effector. Furthermore, the terms “manipulator,” “robotic manipulator,” and “robotic manipulator assembly” are used according to their broad and ordinary meanings, and may refer to a robotic end effector and/or sterile adapter or other adapter component coupled to the end effector, either collectively or individually. For example, the terms “robotic manipulator” and “robotic manipulator assembly” may refer to one or more drive outputs, rails, arms, pulleys, gears, couplings, belts, guides, or the like, whether embodied in a robotic end effector, sterile adapter, and/or other component(s). Robotic manipulators of the present disclosure can include connectors to transfer pneumatic pressure, electrical power, electrical signals, and/or optical signals between the robotic system 10 and a coupled instrument, such as the instrument 19.

The control system 50 can be configured to interface with the robotic system 10, provide information regarding the procedure, and/or perform a variety of other operations. In some examples, the physician 5 can provide input to the control system 50 and/or robotic system 10 via one or more input controls, wherein in response to such input, control signals can be sent to the robotic system 10 to manipulate the instrument 19. The control system 50 can receive real-time images that are captured by the instrument 19 and display the real-time images via the display(s) 56. Additionally, or alternatively, the control system 50 can receive signals (e.g., analog, digital, electrical, acoustic/sonic, pneumatic, tactile, hydraulic, etc.) from a medical monitor and/or a sensor associated with the patient 7, and the display(s) 56 can present information regarding the health or environment of the patient 7.

The robotic system 10 may be communicatively coupled with control system 50 via one or more communication interfaces. The various components of the system 100 can be communicatively coupled to each other over a network, which can include a wireless network and/or a wired network. Example networks include one or more personal area networks (PANs), local area networks (LANs), wide area networks (WANs), Internet area networks (IANs), cellular networks, the Internet, personal area networks (PANs), body area network (BANs), etc. Furthermore, in some embodiments, the various components of the systems can be connected for data communication, fluid exchange, power exchange, and so on via one or more support cables, tubes, or the like.

The control system 50 and/or the robotic system 10 can include certain user controls (e.g., controls 55), which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, and/or interfaces/connectors therefore. Such user controls are communicatively and/or physically coupled to the respective control circuitry. In some embodiments, the user may engage the user controls 55 to command robotic shaft articulation, as described herein.

The instrument 19 (e.g., endoscope) includes a base 11 (e.g., handle) coupled to an elongate shaft 40. The shaft 40 of the instrument 19 can include one or more lights, cameras, and/or sensors (such as pressure sensors and/or other force-reading sensors, which may be configured to generate signals indicating position and/or forces experienced by one or more components of the instrument 19). The shaft 40 can further include one or more working channels, which may run a length of the shaft 40. The instrument 19 also includes one or more mechanisms for causing the shaft 40 to articulate or deflect with respect to an axis thereof. For example, the instrument 19 may include one or more drive inputs that are configured to tension/untension pull wires (not shown for simplicity) to cause articulation of the shaft 40.

In an example use case, if the patient 7 has a kidney stone (or stone fragment) 181 located in a kidney 70, the physician 5 may perform a procedure to remove the stone 181 through the urinary tract (63, 60, 65). In some embodiments, the physician 5 can interact with the control system 50 and/or the robotic system 10 to cause/control the robotic system 10 to advance and navigate the instrument shaft 40 (e.g., a ureteroscope or catheter) from the urethra 65, through the bladder 60, up the ureter 63, and into the renal pelvis 71 and/or calyx network of the kidney 70 where the stone 181 is located. The physician 5 can further interact with the control system 50 and/or the robotic system 10 to cause/control the advancement of a basketing device or other instrument through a working channel of the instrument shaft 40 to facilitate capture and removal of a kidney stone or stone fragment.

As described above, the robotic system 10 includes an articulating medical instrument having an elongate shaft 40 (such as a scope, access sheath, or catheter). In some implementations, the elongate shaft 40 may have a proximal portion with an architecture that is substantially different than an architecture of a distal portion; or even a series of three or more longitudinally staggered portions each having their own unique architecture. Such differing architectures may include those providing different degrees of flexibility, different kinds of articulation (e.g., single-plane articulation vs. two-plane articulation), a dichotomy of articulating vs. non-articulating, or other kinds of differing architecture. Some such elongate instruments may have translating articulation drive features that traverse the full length of the elongate instrument, such that the translating articulation drive features must pass through these longitudinally varying architectures.

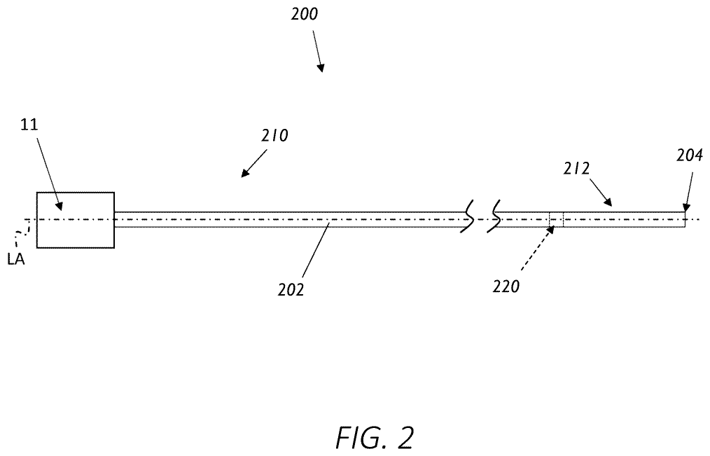

FIG. 2 shows a schematic view of an example elongate instrument 200, according to some implementations. In some implementations, the elongate instrument 200 may be one example of the instrument 19 of FIG. 1. For example, the elongate instrument 200 may be a catheter, scope, access sheath, or other articulable elongate medical instrument.

The elongate instrument 200 of the present example includes a proximal portion 210, a distal portion 212, and a coupling apparatus 220 (also referred to as a “coupler) joining portions 510 and 512 together. In the present example, distal portion 212 is operable to articulate, such that distal end 204 of elongate instrument 200 may be deflected laterally away from and toward a central longitudinal axis (LA) (e.g., defined by proximal portion 210). Further, proximal potion 210 is flexible yet not configured to articulate. In some variations, elongate instrument 200 is operable to articulate at one or more different regions along the length of elongate instrument 200. For example, distal portion 212 may include one or more articulation sections and/or proximal portion 210 may include one or more articulation sections.

The proximal portion 210 includes a base 11 that can be coupled to the robotic system 10, such that robotic system 10 is operable to drive elongate instrument 200 via the base 11. For example, robotic system 10 may be operable to drive translation along the central longitudinal axis LA, rotation (e.g., spinning about LA), articulation, and/or other forms of movement of/by elongate instrument 200. Proximal portion 210 includes a shaft 202 comprising a flexible laser-cut steel hypotube, a braided structure, or any other suitable kind of structure. In some implementations, the shaft 202 may provide primary structural support, while an inner shaft (not shown for simplicity) serves as a liner having a low friction coating (such as polytetrafluoroethylene). In some other implementations, the inner shaft may provide primary structural support, while the outer shaft 202 serves as a liner. Still further, in some implementations, either shaft may comprise a reflow material such as polyether block amide (PEBA) and/or any other suitable kind(s) of material(s).

Distal end 204 of the present example may include one or more openings through which one or more additional instruments may exit into a surgical space or other anatomical region within a patient. Distal end 204 also may include one or more imaging devices that may take the form of one or more cameras and/or one or more optical fibers with corresponding lenses. Distal end 204 also may include one or more illuminating elements, such as one or more integral light-emitting diodes and/or one or more lenses optically coupled with corresponding optical fibers. In some implementations, distal end 204 may include an end effector that is operable to perform one or more operations on tissue, such as grasping, cutting, suturing, sealing (e.g., via RF energy or ultrasonic energy), or stapling, among other examples.

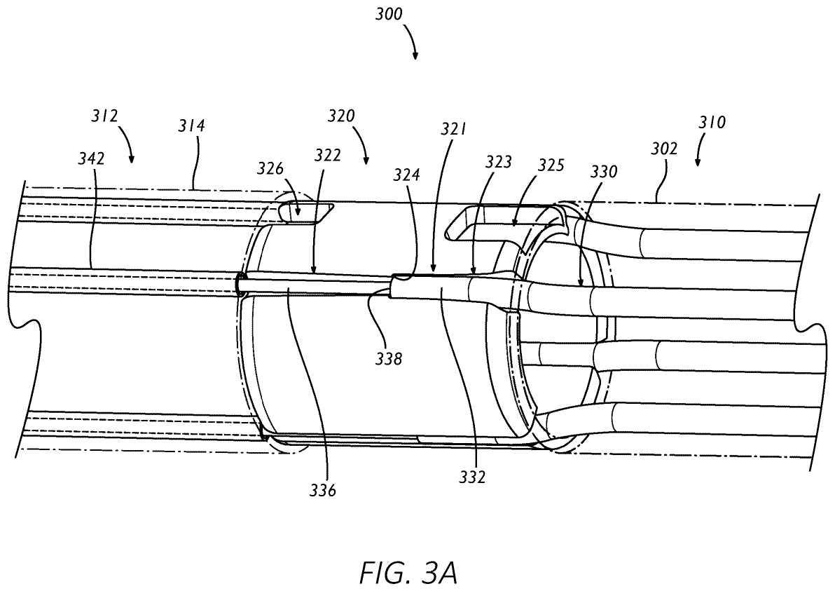

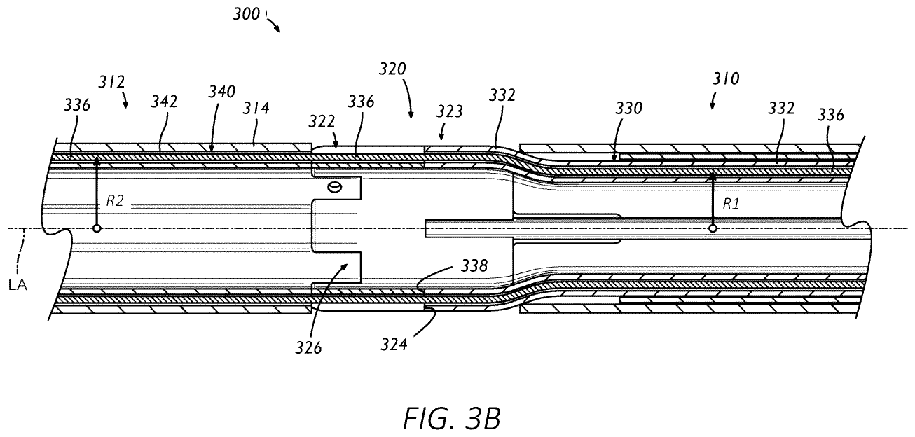

FIG. 3A shows a perspective view of an example elongate instrument 300, according to some implementations. FIG. 3B shows a cross-sectional side view of the elongate instrument 300 shown in FIG. 3A. In some implementations, the elongate instrument 300 may be one example of the elongate instrument 200 of FIG. 2. The elongate instrument 300 includes a proximal portion 310, a distal portion 312, and a coupler 320 joining portions 310 and 312 together. With reference to FIG. 2, the proximal portion 310 may be one example of the proximal portion 210, the distal portion 312 may be one example of the distal portion 212, and the coupler 320 may be one example of the coupler 220.

Proximal portion 310 includes a shaft 302 and proximal tendon assemblies 330. As described with reference to FIG. 2, the shaft 302 may comprise a flexible laser-cut steel hypotube, a braided structure, or any other suitable kind of structure. In the example of FIGS. 3A and 3B, the elongate instrument 300 is shown to include four proximal tendon assemblies 330 that are equidistantly spaced apart (e.g., by 90 degrees). In some other implementations, the elongate instrument 300 may include any number of proximal tendon assemblies having any spacing between them. Each proximal tendon assembly 330 includes a housing 332 and a tendon 336 slidably disposed in a lumen defined by housing 332. Housing 332 is configured to deform laterally yet not compress longitudinally.

Each tendon 336 has a distal end that is fixedly secured at or near a distal end of elongate instrument 300 (such as the distal end 204 of FIG. 2) to provide articulation of distal portion 312. In some implementations, one or more tendons 336 may have a distal end that is fixedly secured at some position that is proximal to distal portion 312. For example, each tendon 336 may comprise a pull wire, a drive band, a single-strand cable, a multi-strand cable, one or more metals, one or more fibers, and/or any other suitable component that is operable to communicate a pulling force along the length of elongate instrument 300, to thereby provide articulation of elongate instrument 300, without substantially stretching.

Distal portion 312 includes a body 314 and distal tendon assemblies 340. Each distal tendon assembly 340 is associated with a corresponding proximal tendon assembly 330, by sharing a common tendon 336. Body 314 may comprise a reflow material such as polyether block amide (PEBA) and/or any other suitable type of material. Each distal tendon assembly 340 includes a housing 342 that defines a lumen having a corresponding tendon 336 disposed therein. Each distal tendon assembly 340 may extend along a path that is parallel with the central longitudinal axis LA of distal portion 312.

Coupler 320 is longitudinally interposed between proximal portion 310 and distal portion 312. As shown in FIGS. 3A and 3B, coupler 320 includes a hollow body that defines a plurality of longitudinally extending channels 321, an array of distal recesses 326, and an array of proximal recesses 325. The hollow configuration of the coupler 320 allows a working channel to pass continuously from proximal portion 310 to distal portion 312 via the coupler 320. In some implementations, coupler 320 may be formed of a rigid material (e.g., molded plastic) as a single, monolithic piece. In some other implementations, coupler 320 may be formed as an assembly of components (e.g., stacked discs). Channels 321 are equidistantly spaced apart (e.g., by 90 degrees) and extend along the full length of coupler 320. Each channel 321 includes a distal portion 322 and a proximal portion 323. Each proximal portion 323 opens through an inner surface of the coupler 320; whereas each distal portion 322 does not open through the inner surface of the coupler 320. A proximally facing shoulder surface 324 is positioned at the transition from proximal portion 323 to distal portion 322.

As shown in FIGS. 3A and 3B, coupler 320 is configured to abut the distal end of proximal portion 310 and the proximal end of distal portion 312. In some implementations, coupler 320 may prevent longitudinal movement of the proximal end of distal portion 312 relative to the distal end of proximal portion 310. In other words, coupler 320 provides a mechanical ground between the distal end of proximal portion 310 and the proximal end of distal portion 312. Proximal portion 323 of each channel 321 is configured to receive a corresponding housing 332 of each proximal tendon assembly 330; while distal portion 322 of each channel 321 is configured to receive a corresponding tendon 336. With housing 332 fitted in proximal portion 323 of channel 321, distal end 338 of housing 332 abuts proximally facing shoulder surface 324. This engagement between distal end 338 of housing 332 and proximally facing shoulder surface 324 provides a mechanical ground between housing 332, coupler 320, distal end of proximal portion 310, and the proximal end of distal portion 312.

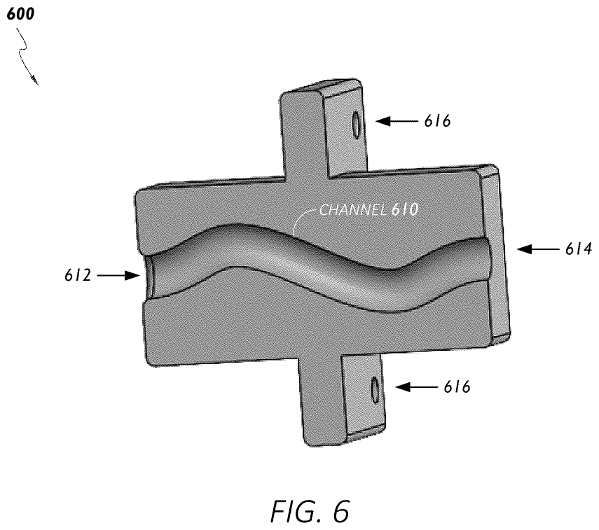

Each tendon 336 is positioned at a first radial distance R1 from the central longitudinal axis LA along proximal portion 310. As shown in FIG. 6, each tendon 336 is positioned at a second radial distance R2 from the central longitudinal axis LA along distal portion 312. In the example of FIGS. 3A and 3B, second radial distance R2 is larger than first radial distance R1. Thus, to transition tendons 336 from proximal portion 310 to distal portion 312, channels 321 guide tendons radially outwardly from the first radial distance R1 to the second radial distance R2. Because each proximal portion 323 of each channel 321 opens through the inner surface of the coupler 320, the distal region of each proximal tendon assembly 330 may bend away from the central longitudinal axis LA and pass through the proximal portion of coupler 320, thereby allowing distal end 338 of housing 332 to engage proximally facing shoulder surface 324; and tendon 336 to pass into distal portion 322 of channel 321.

Distal recesses 326 arc configured to engage with complementary features (e.g., tabs) at the proximal end of distal portion 312; and proximal recesses 325 with complementary features (e.g., tabs) at the distal end of proximal portion 310. In the example of FIGS. 3A and 3B, recesses 325 and 326 and the complementary features of portions 310 and 312 are positioned asymmetrically about the central longitudinal axis LA, such that two of recesses 325 and 326 are angularly separated from each other by 180 degrees in one angular region of coupler 320; and recesses 325 and 326 are angularly separated from each other by 90 degrees in the other angular region of coupler 320. This relationship may provide a consistent, predefined angular positioning between coupler 320 and distal portion 312 about the central longitudinal axis LA. Similarly, proximal recesses 325 are configured to engage with complementary features at the distal end of proximal portion 310, thereby providing a consistent, predefined angular positioning between coupler 320 and proximal portion 310 about the central longitudinal axis LA. It should therefore be understood that coupler 320 may serve as a poke-yoke feature ensuring that proximal portion 310 and distal portion 312 are appropriately aligned with each other angularly about the central longitudinal axis LA.

While three recesses 325 are provided in this example, other variations may include fewer or more recesses 325 than those shown in FIGS. 3A and 3B. The number of complementary features (e.g., tabs, etc.) at the proximal end of distal portion 312 may vary accordingly. Similarly, while three recesses 326 are provided in this example, other variations may include fewer or more recesses 326 than those shown in FIGS. 3A and 3B. The number of complementary features (e.g., tabs, etc.) at the distal end of proximal portion 310 may vary accordingly. While channels 321 of coupler 320 in the present example are all parallel to the central longitudinal axis LA in this example, channels 321 may be oriented differently in other variations. For example, in some implementations, channels 321 may have helical orientations about the central longitudinal axis LA. Such helical orientations of channels 321 may angularly reposition a tendon 336 or other longitudinally extending component, from a first angular position about the central longitudinal axis LA to a second angular position about the central longitudinal axis LA, as the tendon 336 or other longitudinally extending component transitions from proximal portion 310 to distal portion 312.

In some implementations, the coupler 320 may be attached to the proximal portion 310 and/or the distal portion 312 of the elongate instrument 300 via an adhesive. For example, the adhesive may be applied to one or more tabs and/or other surfaces of the proximal portion 310 and/or the distal portion 312 that make contact with the coupler 320. However, applying adhesive to such small surfaces can be time consuming and difficult to control. Thus, in some other implementations, the coupler 320 may be welded to the proximal portion 310 and/or the distal portion 312 of the elongate instrument 300 using a laser. For example, a laser welder can spot-weld the tabs and/or other surfaces of the proximal portion 310 and/or the distal portion 312 that make contact with the coupler 320.

A laser welder uses a highly concentrated beam of light to fuse two (or more) surfaces together. The laser beam must have a very high intensity to generate enough heat for fusing objects together. Even incidental exposure to such high intensity light (such as due to specular or diffuse reflections) can cause damage to the skin and/or eyes. As such, laser welding is often performed inside an enclosure that seals or traps the laser light therein and/or attenuates the intensity of any light transmitted through the enclosure to relatively safe levels. However, a shaft of the elongate instrument 300 (also referred to as a “flexure”) may be longer than the enclosures for many laser welding systems. As such, laser welding must be performed on a portion (such as a proximal end) of the shaft disposed inside the enclosure while a remaining portion (such as a distal end) of the shaft remains outside the enclosure.

Aspects of the present disclosure recognize that a flexure can bend or deform around curves and/or corners that would otherwise occlude or obstruct any incident rays of light. In some aspects, a laser welding enclosure may be coupled to (or include) a light occluding channel that provides a tortuous path through which a flexure can enter or otherwise access the enclosure without allowing any light to leak or otherwise escape through the channel. More specifically, the shape of the light occluding channel (or tortuous path) can be configured to prevent any light within the enclosure from passing through the channel. Accordingly, the light occluding channel can permit a flexure to enter a laser welding enclosure while concurrently blocking any light from exiting the enclosure.

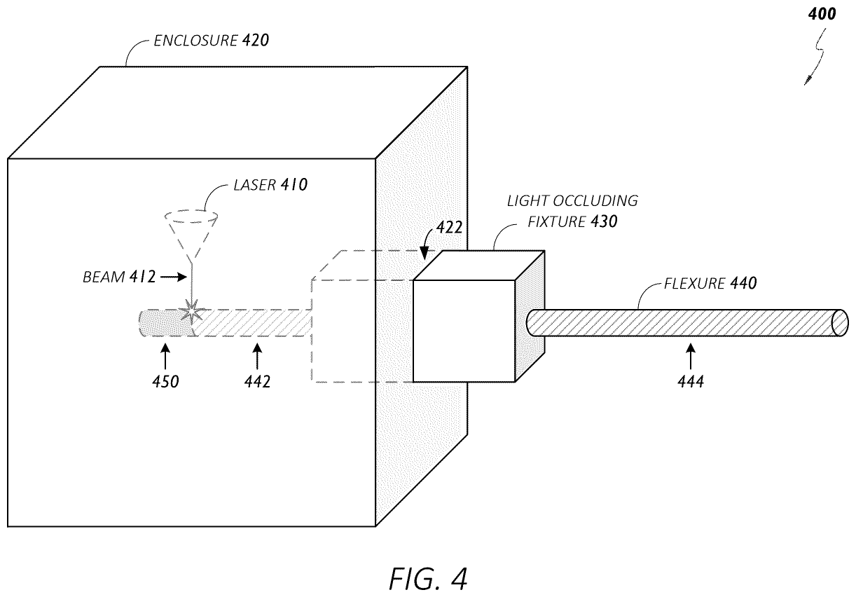

FIG. 4 shows an example laser welding system 400, according to some implementations. The laser welding system 400 can be used to assemble at least a portion of an elongate instrument (such as a catheter, scope, or access sheath). More specifically, the laser welding system 400 uses laser radiation to fuse together two or more segments of the elongate instrument. In the example of FIG. 4, the two or more segments include a flexure 440 and a coupling apparatus 450. In some implementations, the elongate instrument may be one example of the elongate instrument 200 of FIG. 2 or the elongate instrument 300 of FIGS. 3A and 3B. With reference to FIGS. 3A and 3B, the flexure 440 and the coupling apparatus 450 may be examples of the proximal portion 310 and the coupler 320, respectively, of the elongate instrument 300.

The laser welding system 400 includes a laser 410 disposed within an enclosure 420 having an opening to receive the flexure 440. As shown in FIG. 4, the flexure 440 is longer than the enclosure 420 (including any dimension thereof). As a result, only a portion 442 of the flexure 440 (also referred to as the “distal portion”) can be inserted inside the enclosure 420 while a remaining portion 444 of the flexure 440 (also referred to as the “proximal portion”) must remain outside the enclosure 420. The laser 410 is configured to emit a concentrated beam of light 412 (also referred to as a “laser beam”) that can be used to weld the coupling apparatus 450 to the distal portion 442 of the flexure 440. More specifically, the laser beam 412 can be focused or otherwise directed at one or more adjacent and/or overlapping surfaces of the flexure 440 and the coupling apparatus 450. The laser beam 412 transfers energy to the adjacent surfaces in the form of heat, which causes the surfaces to melt and then merge or fuse together.

Light from the laser beam 412 often reflects off the surfaces of the flexure 440 and/or the coupling apparatus 450. As described above, reflected laser light (including specular reflections and/or diffuse reflections) can be harmful to human skin and/or eyes. The enclosure 420 is configured to protect a user or operator of the laser welding system 400 from harmful laser radiation by trapping or sealing the laser light therein. For example, the enclosure 420 may include a number of walls constructed or otherwise formed from opaque and/or translucent materials that block or attenuate the laser light so that any light transmitted through the walls of the enclosure 420 have a wavelength and/or intensity that is safe to human eyes and skin. In the example of FIG. 4, the enclosure 420 is depicted as a rectangular prism or volume. However, in actual implementations, the enclosure 420 may have any shape, size, or volume formed by any number of walls. Because the flexure 440 cannot be fully disposed within the enclosure 420, at least a portion of the enclosure 420 must remain open while performing a laser welding operation so that the distal portion 442 of the flexure 440 can be disposed within the enclosure 420 while the proximal portion 444 of the flexure 440 remains outside the enclosure 420.

In some aspects, the laser welding system 400 may further include a light occluding fixture 430 coupled to a wall of the enclosure 420. More specifically, the light occluding fixture 430 may be disposed within and/or around an opening 422 in the wall of the enclosure 420. In some implementations, the light occluding fixture 430 may be configured to provide access to the enclosure 420 for the flexure 440 while preventing the laser light from escaping through the opening 422. For example, the light occluding fixture 430 may be formed from a solid block of opaque or translucent material that blocks or attenuates the transmission of light, where the block includes a channel through which the distal portion 442 of the flexure 440 can be inserted to enter the enclosure 420. More specifically, the channel is configured to block or occlude the path of any light emitted inside the enclosure 420 so that no reflections of the laser beam 412 can escape through the channel when laser welding the coupling apparatus 450 to the distal portion 442 of the flexure 440. In the example of FIG. 4, the light occluding fixture 430 is depicted as a rectangular prism or volume. However, in actual implementations, the light occluding fixture 430 may have any shape, size, or volume formed by any number of walls.

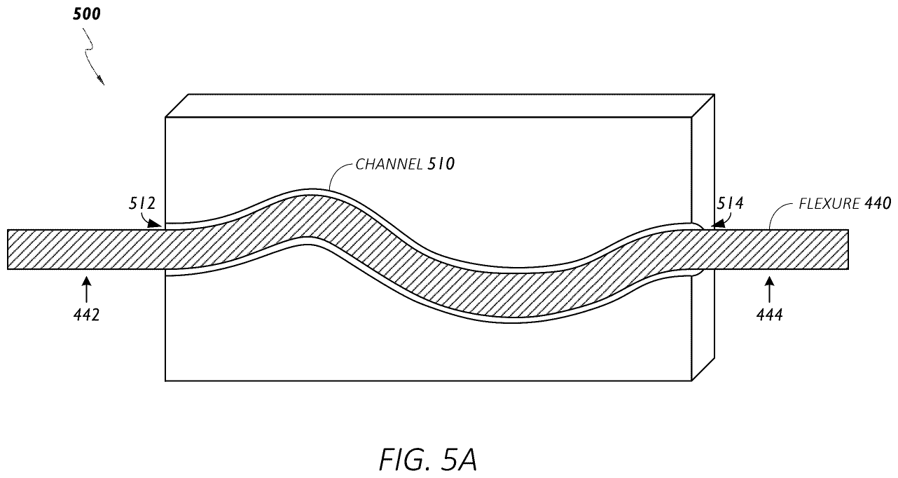

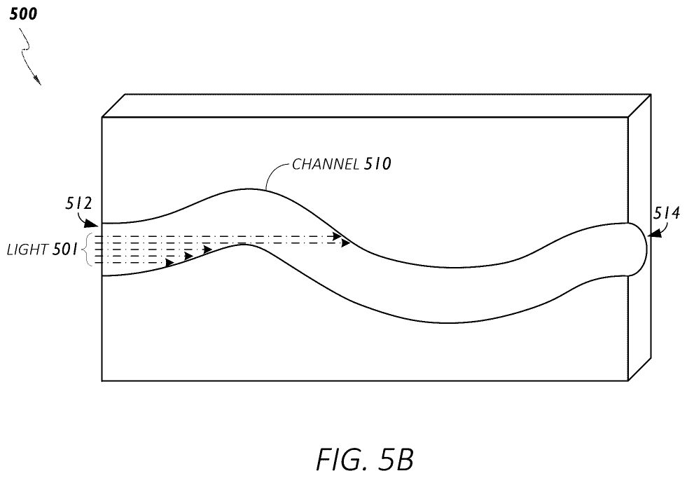

FIGS. 5A and 5B show cross-sectional views of an example light occluding apparatus 500, according to some implementations. In some implementations, the light occluding apparatus 500 may be one example of the light occluding fixture 430 of FIG. 4. With reference for example to FIG. 4, the light occluding apparatus 500 may be coupled to or disposed within a wall of the enclosure 420 (such as via the opening 422). More specifically, FIG. 5A shows the light occluding apparatus 500 with the flexure 440 disposed therein (such as shown in FIG. 4) whereas FIG. 5B shows the light occluding apparatus 500 without the flexure 440.

In the example of FIGS. 5A and 5B, the light occluding apparatus 500 is depicted as a rectangular volume having a first opening 512 and a second opening 514 connected via a channel 510. However, in actual implementations, the light occluding apparatus 500 may have any shape, size, or volume. With reference for example to FIG. 4, the first opening 512 may be positioned inside and/or face toward the enclosure 420 and the second opening 514 may be positioned outside and/or face away from the enclosure 420. In some implementations, the channel 510 may be formed within a solid block of opaque material. In such implementations, no light can be transmitted through any of the walls or surfaces of the light occluding apparatus 500. In some other implementations, the channel 510 may be formed within a solid block of translucent material. In such implementations, the walls or surfaces of the light occluding apparatus 500 may be configured to attenuate incident light so that any light transmitted through such walls or surfaces has a lower intensity and/or frequency.

The channel 510 forms a tortuous path through the light occluding apparatus 500. As used herein, the term “tortuous path” refers to any path that does not follow a straight line (such as a windy or circuitous path). In other words, a tortuous path may have one or more bends, corners, twists, or turns between the beginning of the path and the end of the path. As shown in FIG. 5A, the shape of the channel 510 is configured to allow the flexure 440 to be inserted through the light occluding apparatus 500. More specifically, the distal portion 442 of the flexure 440 can be inserted into the light occluding apparatus 500 via the second opening 514 and exit the light occluding apparatus 500 via the first opening 512. The flexure 440 bends and/or deforms according to the shape of the channel 510 as it traverses the tortuous path between the second opening 514 and the first opening 512. Thus, the curvature of the channel 510 may be configured to support a maximum bend radius or degree of articulation of the flexure 440, thereby allowing the shape of the flexure 440 to conform to the shape of the channel 510 without damaging the flexure 440.

With reference to FIG. 5B, the shape of the channel 510 is also configured to prevent any light 501 incident upon the first opening 512 from exiting through the second opening 514 or vice versa. Although FIG. 5B only shows rays of light 501 that are perpendicular to a surface of the light occluding apparatus 500, the first opening 512 may allow light 501 to enter the channel 510 at various other angles (not shown for simplicity). Any light 501 entering into the channel 510 is blocked or otherwise attenuated by one or more surfaces of the light occluding apparatus 500. For example, as shown in FIG. 5B, a straight line cannot be drawn between the first opening 512 and the second opening 514 without intersecting any surface or boundary of the channel 510. Further, the channel 510 may be configured to reduce or otherwise limit reflections of the light 501. In other words, the inner surfaces of the channel 510 may absorb or attenuate the light 501 entering via the first opening 512 to prevent reflections of the light 501 from exiting the channel 510 via the second opening 514 or to reduce the intensity and/or frequency of any reflected light that exits the channel 510 via the second opening 514.

In some implementations, the inner surfaces of the channel 510 may be formed or treated with a light-absorbing material that prevents reflections of any incident light. In other words, the channel 510 absorbs all the light 501 that enters via the first opening 512 so that none of the light 501 entering the channel 510 can exit via the second opening 514. In some other implementations, the inner surfaces of the channel 510 may be formed or treated with a semi-reflective material that attenuates any reflected light. In other words, with each reflection, the channel 510 absorbs at least some of the light 501 that enters via the first opening 512 so that any reflected light reaching the second opening 514 is effectively nullified or significantly attenuated (such as below a threshold intensity and/or frequency). For example, the tortuous path may include enough bends or curves to ensure that any incident light 501 experiences at least a threshold number of reflections off the inner surfaces of the channel 510 before reaching the second opening 514. The number of reflections may be any number suitable for reducing the intensity and/or frequency of the light 501 to a level safe for exposure to human skin and/or eyes.

Thus, as shown in FIG. 5B, the light occluding apparatus 500 can prevent harmful laser radiation from escaping an enclosure (such as the enclosure 420 of FIG. 4) even when no flexure is disposed within the channel 510. In some aspects, the light occluding apparatus 500 may further include one or more flanges (not shown for simplicity) for attaching or affixing the light occluding apparatus 500 to an enclosure and/or sealing an opening of the enclosure that provides access for the flexure 440 (such as the opening 422 of FIG. 4).

FIG. 6 shows another cross-sectional view of an example light occluding apparatus 600, according to some implementations. In some implementations, the light occluding apparatus 600 may be one example of the light occluding fixture 430 of FIG. 4. With reference for example to FIG. 4, the light occluding apparatus 600 may be coupled to or disposed within a wall of the enclosure 420 (such as via the opening 422).

In the example of FIG. 6, the light occluding apparatus 600 is depicted as a rectangular volume having a first opening 612 and a second opening 614 connected via a channel 610. However, in actual implementations, the light occluding apparatus 600 may have any shape, size, or volume. With reference for example to FIG. 4, the first opening 612 may be positioned inside and/or face toward the enclosure 420 and the second opening 614 may be positioned outside and/or face away from the enclosure 420. In some implementations, the channel 610 may be formed within a solid block of opaque material. In such implementations, no light can be transmitted through any of the walls or surfaces of the light occluding apparatus 600. In some other implementations, the channel 610 may be formed within a solid block of translucent material. In such implementations, the walls or surfaces of the light occluding apparatus 600 may be configured to attenuate incident light so that any light transmitted through such walls or surfaces has a lower intensity and/or frequency

The channel 610 forms a tortuous path through the light occluding apparatus 600. In some implementations, the channel 610 may be one example of the channel 510 of FIGS. 5A and 5B. More specifically, the shape of the channel 610 is configured to allow a flexure (such as the flexure 440 of FIG. 4) to be inserted or threaded through both openings 612 and 614 of the light occluding apparatus 600 (such as described with reference to FIG. 5A). The shape of channel 610 is also configured to prevent any light incident upon the first opening 612 from exiting the channel 610 through the second opening 614 or vice versa (such as described with reference to FIG. 5B). In some implementations, the inner surfaces of the channel 610 may be formed or treated with a light-absorbing material that prevents reflections of any incident light. In some other implementations, the inner surfaces of the channel 610 may be formed or treated with a semi-reflective material that attenuates any reflected light.

In the example of FIG. 6, the light occluding apparatus 600 also includes a flange 616 disposed around an outer surface of the apparatus 600. The flange 616 provides a surface for attaching or securing the light occluding apparatus 600 to a wall of a laser welding enclosure (such as the enclosure 420 of FIG. 4). For example, the flange 616 may include one or more holes (such as shown in FIG. 6) that can be used as attachment points for fastening the flange to a wall of the enclosure. In some implementations, the flange 616 also may be used to seal an opening in the wall of the enclosure through which the light occluding apparatus 600 is at least partially disposed (such as the opening 422 of FIG. 4). With reference for example to FIG. 4, the flange 616 may prevent light emitted within the enclosure (such as reflections of the laser beam 412) from escaping through the opening 422 via gaps between the outer surface of the light occluding apparatus 600 and the wall of the enclosure 420.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A laser welding system, comprising:
an enclosure having an opening to receive an elongate shaft;
a laser configured to emit a concentrated beam of light inside the enclosure; and
a light occluding fixture coupled to the opening of the enclosure, the light occluding fixture being configured to provide access to the enclosure for the elongate shaft and prevent the light from the laser from escaping through the opening while a distal portion of the elongate shaft is disposed within the enclosure and a proximal portion of the elongate shaft is outside the enclosure.

2. The laser welding system of claim 1, wherein the elongate shaft is longer than the enclosure.

3. The laser welding system of claim 1, wherein the elongate shaft comprises a flexure.

4. The laser welding system of claim 1, further comprising:
one or more flanges for sealing the light occluding fixture within the opening of the enclosure.

5. The laser welding system of claim 1, wherein the light occluding fixture comprises:
a first opening facing outside the enclosure;
a second opening facing inside the enclosure; and
a channel connecting the first opening to the second opening, the channel having a shape that prevents any light incident upon the second opening from exiting through the first opening.

6. The laser welding system of claim 5, wherein the channel comprises a tortuous path through the light occluding fixture.

7. The laser welding system of claim 5, wherein the channel provides access to the enclosure for the elongate shaft.

8. The laser welding system of claim 1, wherein the concentrated beam of light is configured to weld the distal portion of the elongate shaft to an attachment.

9. The laser welding system of claim 8, wherein the elongate shaft represents a first segment of a medical instrument.

10. The laser welding system of claim 9, wherein the attachment is a coupling apparatus for coupling the first segment to a second segment of the medical instrument.

11. The laser welding system of claim 10, wherein the medical instrument comprises a catheter and the second segment of the medical instrument is a distal end of the catheter.

12. A light occluding fixture for a laser welding system, comprising:
a first opening configured to face outside an enclosure of the laser welding system;
a second opening configured to face inside the enclosure of the laser welding system; and
a channel connecting the first opening to the second opening, the channel having a shape that prevents any light incident upon the second opening from exiting through the first opening.

13. The light occluding fixture of claim 12, wherein the channel comprises a tortuous path through the light occluding fixture.

14. The light occluding fixture of claim 12, wherein the channel is configured to provide access to the enclosure for an elongate shaft.

15. The light occluding fixture of claim 13, wherein the elongate shaft is longer than the enclosure.

16. The light occluding fixture of claim 13, wherein the elongate shaft comprises a flexure.

17. The light occluding fixture of claim 13, wherein the laser welding system is configured to weld the elongate shaft to an attachment.

18. The light occluding fixture of claim 17, wherein the elongate shaft represents a first segment of a medical instrument.

19. The light occluding fixture of claim 18, wherein the attachment is a coupling apparatus for coupling the first segment to a second segment of the medical instrument.

20. The light occluding fixture of claim 19, wherein the medical instrument comprises a catheter and the second segment of the medical instrument is a distal end of the catheter.
